(19) United States
(12) Patent Application Publication
(10) Pub. No.: US 2006/0214141 A1
(43) Pub. Date: Sep. 28, 2006

(54) Luminescent illumination adjunct for night vision

(76) Inventors: Norbert E. Yankielun, Lebanon, NH (US); Charles M. Reynolds, Lebanon, NH (US)

Correspondence Address: HUMPHREYS ENGINEER CENTER SUPPORT ACTIVITY;ATTN: CEHEC-OC, 7701 TELEGRAPH ROAD, ALEXANDRIA, VA, 22315-3860, US

(21) Appl. No.: 11/086,311

(22) Filed: Mar. 23, 2005

(51) Int. Cl. A61K 31/501; A61K 33/40; C09K 9/00

(52) U.S. Cl. 252/583; 424/616; 514/248

(57) ABSTRACT

A method, and a compound for facilitating it, that enhances night vision by dispersing a luminescent to provide low-intensity area illumination. Luminescents may include naturally occurring bioluminescents (visible spectrum) or man-made, preferably non-toxic, chemical-based luminescents (also termed chemiluminescents), the latter available for use in either the visible or IR spectrum. It may be applied locally to a surface or remotely by means of a delivery system. Preferably, select luminescents are dispersed as an aerosol to contact targeted surfaces. These luminescents may be used in spaces otherwise difficult to image with night vision equipment. Specifically provided is a method for viewing a target under low ambient light conditions comprising dispersing a luminescent material on surfaces in a dark space to provide a low-level, spatially broad, source of supplemental scene illumination, and viewing the target with image enhancing devices that are otherwise marginally useful without the presence of the luminescent material.

### STATEMENT OF GOVERNMENT INTEREST

Under paragraph 1(a) of Executive Order 10096, the conditions under which this invention was made entitle the Government of the United States, as represented by the Secretary of the Army, to the entire right, title and interest therein of any patent granted thereon by the United States. This patent and related ones are available for licensing. Contact Sharon Borland at 703 428-9112 or Phillip Stewart at 601 634-4113.

### BACKGROUND

A number of definitions exist for the term “luminescence” and variants thereof. In general the term may be used to refer to an emission of light that is not directly ascribable to incandescence and, therefore, occurs at low temperature. Luminescence may originate from physiological or biological processes (bioluminescence), chemical reactions (chemiluminescence), and friction. Some define the term to include the production of light by electrical action, cathode rays and light, while others do not. For example, Honeywell's LUMILUX® pigment absorbs and stores energy from light, then emits it for up to eight hours after an energy source is removed. It is commonly used in markers that help guide building occupants through rooms, hallways and stairwells during emergencies, such as fire or power failure. The terms “luminescence,” “luminescent,” and variants thereof, as used herein, refer to light produced by chemical or biological processes, i.e., chemiluminescence and bioluminescence, respectively.

Bioluminescence is a naturally occurring phenomenon that is relatively common. Visible light-emitting systems have been known and isolated from many luminescent organisms including bacteria, protozoa, coelenterates, mollusks, fish, millipedes, flies, fungi, worms, crustaceans, and beetles, particularly click beetles of genus Pyrophorus and the fireflies of the genera Photinus, Photuris, and Luciola. In many of these organisms, enzymes catalyze monooxygenations and utilize the resulting free energy to excite a molecule to a high energy state. Visible light is emitted when the excited molecule spontaneously returns to the ground state. This emitted light is called “bioluminescence.”

Varieties of plankton, krill, and other salt water and freshwater aquatics are known to produce light in the visible spectrum. Several members of the insect community have similar capabilities, e.g., fireflies, glow worms, etc. Fireflies produce light via a chemical reaction consisting of Luciferin (a substrate) combined with Luciferase (an enzyme), ATP (adenosine triphosphate) and oxygen. When these components are added, light is produced. The reaction is described as:
luciferin+luciferase+ATP→luciferyl adenylate-luciferase+pyrophosphate luciferyl adenylate-luciferase+O₂→oxyluciferin+luciferase+AMP+light
McElroy, W. D., Properties of the Reaction Utilizing Adenosine Triphosphate for Bioluminescence, J. Biol. Chem., 191:547-557, 1951.

Some varieties of fungi are also known to produce low levels of visible illumination. A common term for luminescent fungi is “foxfire”. These fungi are relatively innocuous, feeding on decaying organic material typically found on forest floors. Spores of applicable species of fungus may be grown commercially, harvested and appropriately prepared and packaged for dispersal. Certain environmental conditions are required for the fungi to thrive, and there is some delay between dispersal of these spores and the time when the fungi sufficiently develop to produce bioluminescence. It may be possible to package desiccated minute particles of the mature form of these fungi as a powder suitable for dispersal. It is possible that these desiccated mature fungi may begin to bioluminesce on an accelerated schedule if environmental conditions are amenable. Since these are naturally occurring species that are common in most ecosystems worldwide, there should be little or no negative impact on their introduction into the environment. Since they exist on decaying organic material, once their source of nutrients is consumed, they are “extinguished.”

Armillaria mellea and a closely related relative are common root rot and wood decay fungi found across North America, Europe and Asia. Armillaria grows in (and on) old stumps, dead trees, buried roots, and downed logs. The fruiting body of Armillaria is a small golden-colored, stalked mushroom. This fruiting body is not luminescent. Armillaria's mycelium and rhizomorphs are luminous. The root-like dark rhizomorphs, when they stop growing or when entering a resting period lose luminosity.

The most actively growing and respiring fungal cells generate light. The conditions that allow the fungi to grow fast, allow light to be produced. The most important environmental features surrounding fungal bioluminescence is food supply followed closely by water, oxygen, and temperature.

The cell wall components and remains of sugars, starch, and proteins in the wood are the desired food-stock of Armillaria. The luminescence can last in one piece of wood for up to eight weeks until essential resources are consumed. It usually takes at least four weeks to build to maximum luminescence.

The rotting wood must be kept moist. If it is too dry, fungal growth stops. If it is too wet fungal growth is suffocated. Moisture is an important feature of luminescent wood because the process of light generation produces water as a by-product. Luminescent wood feels saturated. The glowing, rotting pieces of wood need to be kept moist, not soaked.

Oxygen is critical to keep the fungi healthy and growing. Too much water can make oxygen movement more difficult, and light generation will decline and be extinguished.

The optimum temperature for Armillaria bioluminescence is 77° F. (25° C.). Light generation is noticeable as low as 34° F. (1° C.). Light generation declines rapidly and stops above 86° F. (30° C.).

Maximum light is achieved under acid conditions (pH 5.7-6.0). Presence of the ammonium form of nitrogen allows for more energy to be released as light.

The fungi generate light over an 80-nm range, equivalent to one-fourth the width of the entire visible spectrum (˜400-700 nm). Most of the light generated is in a narrow band within that range. The maximum light output occurs at a wavelength peak of 520-530 nm. The 525 nm peak wavelength is about 40 nm shorter than firefly light and 50 nm longer than luminescence bacteria. That is, the luminous glow is emitted in the bluish-green portion of the spectrum. The color seen in nature can be slightly different because of the wood and dirt the light is filtered through. Darker, older cell walls and surface layers will change the color of the light showing through. The color you see is also affected by an individual's color vision at night. Color descriptions range from a stark blue to a sickly green.

Armillaria bioluminescence has a daily light intensity rhythm with maximum intensity around 7:30 PM and a minimum intensity around 7:30 AM. The light intensity rhythm is not affected by total darkness, total light, or changing daily light periods.

Man-made chemical alternatives employing the phenomenon of chemiluminesence (CL), are an alternative to bioluminescence as visible illumination for night vision enhancement. A CL technique employs the use of a CL reagent such as luminol (5-amino-2,3-dihydro-1,4-phthalazinedione) in the presence of an oxygen donor reactant, such as hydrogen peroxide, which upon contact with biological materials produces detectable light via chemiluminesence. Generic biological components, e.g., grasses and other vegetation, may initiate the CL-based reaction.

Commercial sources of non-toxic, moderate persistence (8-24 hour) chemical-based luminescent materials have been available for over two decades. Typically, light intensity of these chemical sources is proportionate to ambient temperature. In a warmer environment greater light intensity is produced. Light duration time is inversely proportional to temperature. The colder the environment, the longer the illumination persists. Thus, in a warm environment there is relatively high intensity light for relatively short duration and in a cool environment there is relatively low intensity light for relatively long duration.

One commercial class of product employs a binary chemical that has excellent long-term storage stability, providing a non-thermal source of visible light. The commercial name of one such product is CYALUME®, manufactured by Omniglow Corporation. It consists of two non-toxic chemicals, bis(2,4,5-trichlorophenyl-6-carbopentoxyphenyl)oxalate (CPPO) with hydrogen peroxide, that when mixed produces non-toxic, non-exothermic, chemical illumination that persists for 8 to 24 hours, depending on environmental conditions. Typically fluorescent dyes, or fluorophors, are added to the two-component system to add color to the chemiluminescent product. Other peroxyoxalate esters, such as bis(2,4,6-trichlorophenyl)oxalate (TCPO) and [bis(2-(3,6,9-trioadecanyloxycarbonyl)-4-nitrophenyl]oxalate (TDPO), react with hydrogen peroxide in a similar manner to produce chemiluminescence. Luminol (or its derivatives) may also be used as a chemiluminescent in combination with hydrogen peroxide or other oxidants and a cation catalyst. Other common or well known solution phase systems that may be used in the invention include lucigenin (or its derivatives), ruthenium tris-bipyridine, and luciferin.

Night vision devices (monoculars, binoculars, telescopes) are essentially light intensifiers or “light amplifiers” providing the ability to view scenes having a minimum amount of ambient lighting. These devices typically employ available low level ambient visible light sources, such as moonlight or starlight, to clearly view scenes that to the naked eye appear blacked out or too darkened for resolution, recognition and identification of objects of interest, such as individuals, terrain features, infrastructure and vehicles. Conventional night vision devices are sensitive to photonic energy in the visible and near infrared portion of the electromagnetic spectrum. Even the latest generation night vision devices require at least a minute amount of ambient lighting to enable a user to recognize objects in an otherwise darkened environment. Under certain circumstances there may not be sufficient ambient light for the devices to work. Possible environments where night vision devices may become partially or fully ineffective include: densely canopied forests, irregular landscape with ravines and gullies where natural ambient starlight or moonlight is restricted, e.g., shadowed or blocked by the topography. Locations where night vision may be less than effective include urban environments where man-made structures cause shadowing from ambient light. The interior of buildings, basements, caves, narrow streets and alleys between high rises, and tunnels may lack sufficient ambient lighting to permit effective use of night vision equipment.

Ground-fired and parachute flares provide a high intensity point source of night vision enhancement, but still leave shadowy regions. The light from the flares may be so intense as to desensitize a night vision devise, leading to decreased imaging efficiency.

Night vision imaging ability may be improved by using a visible point source of moderate intensity such as a flashlight or spotlight. Moderate and high intensity visible lighting has the obvious shortcomings of lack of stealth, i.e., ease of detection of its use, as well as potential for pinpointing the user's position. Early attempts to covertly supplement ambient lighting to aid in target recognition with night vision relied on the use of a point source of infrared illumination. This concept was used as early as the Korean conflict and still is a common solution, especially with early Generation I and II night vision equipment, and to a lesser extent, with the latest equipment.

There are several advantages to using infrared illumination to supplement ambient lighting for night applications. Infrared illumination is not visible to the unaided human eye. An adversary without night vision equipment will not have the advantage of the illumination. There are also drawbacks to the use of an infrared illuminator. It is yet another piece of equipment that has to be carried and operated by personnel. In the case of an adversary equipped with night vision capability, not only is this illumination visible, it also locates the operator of the illuminator.

An alternative, disposable, point source infrared illuminator is implemented with IR light emitting diodes (LED's) and batteries. The diodes may be provided singly or as an array. Such an illuminator may be emplaced into a dark environment and provide a point source of illumination capable of localized illumination. This is a fairly simple and economical alternative. There are several disadvantages, however, including that it is only a point source and has limited spatial illumination capability. Once discovered by an adversary, it could be easily destroyed or cloaked, eliminating its usefulness.

Similarly, chemical luminescent devices, such as CYALUME LIGHT STICKS® (Omniglow Corp.) if distributed throughout a scene may provide background lighting in the visible or infrared spectrum. These point sources of chemical luminescent light may also be destroyed or cloaked by an adversary, rendering them ineffective.

Any point or distributed source of light that is of sufficient intensity to be generally visible to an adversary reveals that they may be under observation. Additionally, a point source of illumination still may not fully illuminate a volume. Given the local topography, there may be dark or “blind” shadowed features where conventional passive night vision equipment may not “penetrate” the darkness efficiently or clandestinely.

Thus, what is needed is an alternative means of providing a low-level, spatially broad, source of supplemental scene illumination that permits passive viewing using night vision devices. Such a capability is provided in embodiments of the present invention.

### DETAILED DESCRIPTION

Provided is a method for enhancing night vision by atomizing and dispersing a photoluminescent material to provide low-intensity area illumination. This material may be a naturally occurring bioluminescent (visible light) or a man made non-toxic chemical-based luminescent (visible or IR light). It may be applied directly or dispersed remotely by means of a delivery system. It may be used in environments or volumes otherwise difficult to image with night vision equipment. Unlike a conventional point source of illumination, an aerosol low-level illumination source conformally coats a complex geometric environment leaving few, if any dark zones or shadowed features.

In select embodiments of the present invention, a method for enhancing night vision in an area lacking sufficient ambient light for use of night vision devices comprises providing one or more luminescent compounds dispersed over at least part of a dark or un-illuminated area of interest, so that the area becomes viewable with a night vision device for at least a pre-specified period.

In select embodiments of the present invention, the method disperses the compound as an aerosol. In select embodiments of the present invention, the method provides the aerosol as two separate constituents, mixing the separate constituents prior to dispersal as an aerosol.

In select embodiments of the present invention, the constituents are non-toxic chemicals. In select embodiments of the present invention, the chemicals are luminol (5-amino-2,3-dihydro-1,4-phthalazinedione) and an oxygen donor reactant. In select embodiments of the present invention, the reactant is hydrogen peroxide.

In select embodiments of the present invention, the method provides the aerosol as two separate constituents, mixing the separate constituents concurrently with dispersal as an aerosol. In select embodiments of the present invention, the method provides these concurrently mixed constituents as non-toxic chemicals. In select embodiments of the present invention, the concurrently mixed chemicals are luminol (5-amino-2,3-dihydro-1,4-phthalazinedione) and an oxygen donor reactant. In select embodiments of the present invention, the reactant used as one of the concurrently mixed chemicals is hydrogen peroxide.

In select embodiments of the present invention, the method employs an aerosol comprising one or more chemiluminescents. In select embodiments of the present invention, one or more of the Chemiluminescents are commercial-off-the-shelf (COTS) products.

In select embodiments of the present invention, the method provides for mixing one or more commercial-off-the-shelf (COTS) products with one or more diluents. In select embodiments of the present invention, water is a diluent.

In select embodiments of the present invention, a COTS chemiluminescent is mixed with a diluent in a ratio of approximately between approximately ten parts of diluent to one part of COTS chemiluminescent and fifty parts of diluent to one part of COTS chemiluminescent. In select embodiments of the present invention as immediately above, the diluent is water.

In select embodiments of the present invention, a COTS chemiluminescent is mixed with a diluent in a ratio of approximately thirty-two parts of diluent to one part of COTS chemiluminescent. In select embodiments of the present invention as immediately above, the diluent is water.

In select embodiments of the present invention, at least one of the luminescent utilized is a bioluminescent material. In select embodiments of the present invention, one or more of the bioluminescent materials is provided as a naturally occurring organism.

In select embodiments of the present invention, the method one or more of the naturally occurring organism is a bioluminescent flora. In select embodiments of the present invention, the bioluminescent flora used is one or more fungi. In select embodiments of the present invention, a nutrient is affixed to the fungi. In select embodiments of the present invention, the fungi is provided as one or more white-spored Basidiomycetes. In select embodiments of the present invention, the fungi are provided as Armillaria mellea.

In select embodiments of the present invention, a method is provided for facilitating viewing of a dark space, including items therein, comprising providing a luminescent material on at least a part of one surface of the space such that the material provides a spatially broad source of illumination and viewing the area with image enhancing devices, such that utility of the devices is enhanced by the dispersed luminescent material.

In select embodiments of the present invention, the luminescent material used in the immediately above method is atomized prior to dispersal, preferably immediately prior.

In select embodiments of the present invention, the luminescent material used with the immediately above method is one or more chemiluminescent materials.

In select embodiments of the present invention, atomized luminescent material is hand dispersed. In select embodiments of the present invention, the luminescent material is dispersed remotely via a delivery system. In select embodiments of the present invention, one or more airborne vehicles are employed as the delivery system. In select embodiments of the present invention, one or more explosive devices are employed as a delivery system. In select embodiments of the present invention, one or more ballistic devices are employed as a delivery system.

In select embodiments of the present invention, the luminescent material is provided in micro-spheres.

In select embodiments of the present invention, luminescent material is provided as one or more bioluminescent materials. In select embodiments of the present invention, the bioluminescent material is selected from substances found in the group of naturally occurring at least partially luminescent materials consisting essentially of: plankton, krill, fungi, bacteria, protozoa, coelenterates, mollusks, fish, millipedes, flies, fungi, worms, crustaceans, beetles, and combinations thereof.

In select embodiments of the present invention, the beetles are click beetles of genus Pyrophorus and the flies are fireflies of the genera Photinus, Photuris, and Luciola.

In select embodiments of the present invention, the method provides for a delay in growth of the bioluminescent material after dispersal thereof.

In select embodiments of the present invention, the method provides for tagging of items via dispersal of the luminescent material. In select embodiments of the present invention, the method provides for tagging by direct dispersal of the luminescent material upon the items.

In select embodiments of the present invention, the method provides for viewing disturbances upon the surfaces in contact with the dispersed luminescent material, such that the disturbances provide an indication of intrusion in the space of interest during a pre-specified time period.

In select embodiments of the present invention, the luminescent material contains an additive that imparts a detectable change in the luminescent material in the presence of a particular substance in the space that contains the dispersed luminescent material.

In select embodiments of the present invention, provided is a compound suitable for providing broad spatial illumination of a space in which the compound is dispersed to contact one or more surfaces thereof. The compound comprises one or more chemiluminescents and one or more diluents added to the chemiluminescents, such that addition of the diluents facilitates dispersing the chemiluminscent as an aerosol.

In select embodiments of the present invention, one or more of the Chemiluminescents in the compound are COTS products. In select embodiments of the present invention, the compound employs water as a diluent.

In select embodiments of the present invention, the compound contains one or more COTS products mixed with diluent in a ratio of approximately between approximately ten parts diluent to one part COTS chemiluminescent and approximately fifty parts diluent to one part COTS chemiluminescent.

In select embodiments of the present invention, the compound contains one or more COTS product mixed with a diluent in a ratio of approximately thirty-two parts diluent to one part COTS chemiluminescent.

Select embodiments of the present invention provide a method for enhancing night vision in an area or volume lacking sufficient ambient light for use of night vision devices. An embodiment of a method of employment of the present invention comprises providing at least one luminescent compound as an aerosol and dispersing the luminescent material over at least part of an area of interest, such that part of the area becomes viewable with a night vision device for at least a pre-specified period.

In select embodiments of the present invention, the method provides the compound as two separate constituents, mixing the separate constituents upon dispersal as an aerosol.

In select embodiments of the present invention, the method provides the compound as two separate constituents, mixing the separate constituents immediately prior to dispersal as an aerosol.

In select embodiments of the present invention, liquid solutions of varying phase duration may be implemented for different applications. In other embodiments of the present invention, dry or powder-based formulations may be used. Select embodiments of the present invention envision use in scenarios in which the luminescent material is dispersed over solid surfaces. Other embodiments of the present invention envision applications in which the luminescent is applied to small still bodies of water. Over water dispersal may be effected in the same manner as for applications on solid surfaces if the luminescent is appreciably water insoluble. If there is any significant water solubility, to provide “water buoyancy,” the luminescent may be blended with a hydrophobic material, such as an oil-based additive suitable for aerosol dispersal. Select embodiments of the present invention may be implemented using micro-spheres. The luminescent material is microencapsulated within a frangible shell which may provide a “tamper-proof” implementation since the micro-spheres remain intact and dormant after dispersal and only after they are physically disturbed (broken) by means of physical contact do they begin to luminesce. Select embodiments of the present invention employing micro-spheres may be used to provide a time-delay by formulating with two components separated by a shell made of a material that slowly dissolves in the targeted dispersing medium, e.g., water.

Select embodiments of the present invention may disperse luminescent material at any concentration and coverage rate sufficient to permit illumination and viewing of an environment otherwise difficult to image with current generation night vision equipment. This may include a concentration and coverage rate to permit sufficient illumination and viewing of such an environment by the naked eye, i.e., without the use of supplemental equipment such as night vision equipment. Other an embodiments of the present invention may be dispersed so as to maintain clandestine surveillance, dispersing the luminescent at a concentration and coverage rate permitting illumination sufficient for use with current generation night vision equipment only.

In select embodiments of the present invention, the method provides the constituents as non-toxic chemicals. In select embodiments of the present invention, the method provides the chemicals as luminol (5-amino-2,3-dihydro-1,4-phthalazinedione) and at least one oxygen donor reactant. In select embodiments of the present invention, the method provides the reactant as hydrogen peroxide.

In select embodiments of the present invention, the method provides the aerosol as a chemiluminescent. In select embodiments of the present invention, the chemiluminescent is derived from a commercial-off-the-shelf (COTS) product.

In select embodiments of the present invention, the method provides the luminescent aerosol as bioluminescent material. In select embodiments of the present invention, the method provides the bioluminescent material as a naturally occurring organism. In select embodiments of the present invention, the method provides the naturally occurring organism as bioluminescent flora. In select embodiments of the present invention, the method provides the bioluminescent flora as at least one fungus. In select embodiments of the present invention, the method provides the fungi affixed to a nutrient therefor. In select embodiments of the present invention, the fungi are white-spored Basidiomycetes.

In select embodiments of the present invention, atomized luminescents may be applied locally by direct manual spraying from an atomizer or fogger. In select embodiments of the present invention, wider area dispersion may employ a grenade or a hand-held explosively dispersive device, such as a paint ball gun. In select embodiments of the present invention, the luminescent may be delivered ballistically to remote or denied areas by means of a mortar or artillery shell, rocket, or air-dropped munitions. In select embodiments of the present invention, the luminescent may be sprayed over a wide area in a “crop-duster” fashion by means of manned- or remotely-piloted aircraft.

While the most obvious application of an embodiment of the present invention is illumination of regions that are otherwise opaque to night vision sensors, select embodiments of the present invention may provide for detection of tampering and intrusion as well as facilitating tracking of targets. For example, a chemiluminescent that is formulated as a powder and delivered as an aerosol may be employed to indicate tampering or intrusion. The powder, not visible to the unaided eye prior to being disturbed, e.g., abraded or crushed, would “luminesce” upon coming in contact with an intruder. In select embodiments of the present invention, a may luminescent may be formulated to stick to the shoes and clothing of an intruder. An intruder's footprints, for example, appear when “luminescent-protected” flooring is viewed using night vision equipment. In select embodiments of the present invention, a room or other area is treated as above and subsequently checked at night using night vision equipment for signs of tampering or intrusion.

In select embodiments of the present invention, a dispersed luminescent may be formulated to adhere to personnel or equipment. Thus, assets “tagged” directly in this manner are self-illuminating. In select embodiments of the present invention, tagging of assets may be performed directly or indirectly. In select embodiments of the present invention, direct tagging introduces the luminescent by directly dispersing it upon them. In select embodiments of the present invention, indirect tagging is accomplished by dispersing a properly formulated luminescent onto terrain over which target assets move. In select embodiments of the present invention, a luminescent may assist a monitoring effort by adhering to assets upon contact of the asset with the “treated” terrain or by indicating a disturbance of the terrain that shows movement across it by the asset. In select embodiments of the present invention, coded luminescents employing color, wavelength, or mixed colors, and the like to encode a “marker,” may provide detailed information on movement and marshalling of specific assets. In select embodiments of the present invention, by employing a color coded illuminating medium, such as by adding a dye as is done with CYALUME® or by chemically introducing a colored moiety into a chemiluminescent molecule, movement and combining of multiple adversary assets may be more easily ascertained.

In select embodiments of the present invention, an additional chemical capability is provided by coupling a luminescent source with a “litmus test” or “dye marker” capability. That is, a “coupled addition” permits the distributed luminescent agent to be initiated or extinguished by environmental or scene conditions. In select embodiments of the present invention, this may include indicating the presence of human activity, e.g., the presence of urine, an increase in carbon dioxide levels, an increase in methane levels, thermal differentials in an area or scene, etc. In select embodiments of the present invention, vehicular activity may be detected by the increase in hydrocarbon products in the surrounding environment. In select embodiments of the present invention, coupling may be accomplished chemically as described immediately above. In select embodiments of the present invention, coupling may be accomplished by the introduction of a dye moiety into a chemiluminescent molecule. In select embodiments of the present invention, the environmental marker may be coupled with the luminescent materials in micro-spheres.

### EXAMPLE

A closed, windowless room was examined initially both with the naked eye and with Generation III night vision goggles while lights were turned off to determine that there was insufficient ambient light in the room to perceive any objects by either means. The surface of a white wall was then sprayed from a distance of about one meter with a chemiluminescent material solution prepared by breaking a CYALUME® stick and diluting its contents of approximately one ounce of liquid with thirty two ounces of water. The lights were then shut off and the room was again viewed with the naked eye and with Generation III night vision goggles. No illumination was perceived with the naked eye, while images of objects in the room could be discerned with the use of the night vision goggles.

In addition to obvious military and national security applications, such as border security, embodiments of the present invention may be used in law enforcement, surveillance, search and rescue, and in maintaining local perimeters such as may be used in commercial settings.

While the invention has been described in terms of its preferred embodiments, those skilled in the art will recognize that the invention may be practiced with modifications within the spirit and scope of the appended claims. For example, although the system is described in specific examples for fungi, it is amenable for use with other bioluminescent material. Thus, it is intended that all matter contained in the foregoing description shall be interpreted as illustrative rather than limiting, and the invention should be defined only in accordance with the following claims and their equivalents.

The abstract is provided to comply with the rules requiring an abstract, which will allow a searcher to quickly ascertain the subject matter of the technical disclosure of any patent issued from this disclosure. It is submitted with the understanding that it will not be used to interpret or limit the scope or meaning of the claims. 37 CFR § 1.72(b). Any advantages and benefits described may not apply to all embodiments of the invention.

We claim:

1. A method for enhancing night vision in an area lacking sufficient ambient light for use of night vision devices, comprising:
providing at least one luminescent compound;
dispersing said luminescent material over at least part of said area, wherein said part of said area becomes viewable with a night vision device for at least a pre-specified period.

2. The method of claim 1 providing said compound as an aerosol.

3. The method of claim 2 providing said aerosol as two separate constituents, mixing said separate constituents prior to dispersal as said aerosol.

4. The method of claim 3 providing said constituents as non-toxic chemicals.

5. The method of claim 3 providing said chemicals as luminol (5-amino-2,3-dihydro-1,4-phthalazinedione) and an oxygen donor reactant.

6. The method of claim 5 providing said reactant as hydrogen peroxide.

7. The method of claim 2 providing said aerosol as two separate constituents, mixing said separate constituents concurrently with dispersal as said aerosol.

8. The method of claim 7 providing said constituents as non-toxic chemicals.

9. The method of claim 7 providing said chemicals as luminol (5-amino-2,3-dihydro-1,4-phthalazinedione) and an oxygen donor reactant.

10. The method of claim 9 providing said reactant as hydrogen peroxide.

11. The method of claim 2 providing said aerosol as at least one chemiluminescent.

12. The method of claim 11 providing at least one said chemiluminescents as a commercial-off-the-shelf (COTS) product.

13. The method of claim 12 mixing at least one said commercial-off-the-shelf (COTS) products with at least one diluent.

14. The method of claim 13 providing water as at least one of said diluents.

15. The method of claim 13 mixing said COTS chemiluminescent with said diluent in a ratio of approximately between approximately ten parts said diluent to one part said COTS chemiluminescent and fifty parts said diluent to one part said COTS chemiluminescent.

16. The method of claim 13 mixing said COTS chemiluminescent with said diluent in a ratio of approximately thirty-two parts said diluent to one part said COTS chemiluminescent.

17. The method of claim 1 providing said luminescent as bioluminescent material.

18. The method of claim 17 providing said bioluminescent material as a naturally occurring organism.

19. The method of claim 18 providing said naturally occurring organism as bioluminescent flora.

20. The method of claim 19 providing said bioluminescent flora as at least one fungus.

21. The method of claim 20 providing said fungi affixed to a nutrient therefor.

22. The method of claim 20 providing said fungi as a white-spored Basidiomycetes.

23. The method of claim 20 providing said fungi as Armillaria mellea.

24. A method for facilitating viewing of a dark space, including items therein, comprising:
providing a luminescent material on at least a part of one surface of said space, wherein said material provides a spatially broad source of illumination; and
viewing said area with image enhancing devices, wherein utility of said devices is facilitated by said dispersed luminescent material.

25. The method of claim 24 atomizing said luminescent material prior to dispersal.

26. The method of claim 25 providing said luminescent material as at least one chemiluminescent material.

27. The method of claim 25 by hand dispersing said atomized luminescent material.

28. The method of claim 24 remotely dispersing said luminescent material via a delivery system.

29. The method of claim 28 providing an airborne vehicle as said delivery system.

30. The method of claim 28 providing an explosive device as said delivery system.

31. The method of claim 28 providing a ballistic device as said delivery system.

32. The method of claim 24 providing said luminescent material in micro-spheres.

33. The method of claim 24 providing said luminescent material as at least one bioluminescent material.

34. The method of claim 33 providing said bioluminescent material selected from substances found in the group of naturally occurring at least partially luminescent materials consisting essentially of: plankton, krill, fungi, bacteria, protozoa, coelenterates, mollusks, fish, millipedes, flies, fungi, worms, crustaceans, beetles, and combinations thereof.

35. The method of claim 34 providing said beetles as click beetles of genus Pyrophorus and said flies as fireflies of the genera Photinus, Photuris, and Luciola.

36. The method of claim 33 further providing for a delay in growth of said bioluminescent material after dispersal thereof.

37. The method of claim 24 further providing tagging of at least one said items via said dispersal of said luminescent material.

38. The method of claim 37 further providing said tagging by direct dispersal of said luminescent material upon said items.

39. The method of claim 24 further providing for viewing disturbances in said dispersed luminescent material,
wherein said disturbances provide an indication of intrusion in said space at least during a pre-specified time period.

40. The method of claim 24 combining with said luminescent material an additive that imparts a detectable change in said luminescent material in the presence of a particular substance in said space.

41. A compound suitable for providing broad spatial illumination of a space in which said compound is dispersed to contact at least some surfaces thereof, comprising:
at least one chemiluminescent; and
at least one diluent added to said chemiluminescents,
wherein addition of said diluent facilitates dispersing said chemiluminscent as an aerosol.

42. The compound of claim 41 in which at least one said chemiluminescent is a COTS product.

43. The compound of claim 42 in which at least one of said diluents is water.

44. The compound of claim 42 in which at least one said COTS product is mixed with said diluent in a ratio of approximately between approximately ten parts said diluent to one part said COTS chemiluminescent and approximately fifty parts said diluent to one part said COTS chemiluminescent.

45. The compound of claim 42 in which at least one said COTS product is mixed with said diluent in a ratio of approximately thirty-two parts said diluent to one part said COTS chemiluminescent.
